(19) United States
(12) Patent Application Publication
(10) Pub. No.: US 2008/0287931 A1
(43) Pub. Date: Nov. 20, 2008

(54) Use of iontophoresis and ultrasound to deliver melanin or other chromophores for laser hair removal

(76) Inventors: Dennis R. Jones, Chesapeake, VA (US); Evan D. Parker, Virginia Beach, VA (US)

Correspondence Address: GUERRY LEONARD GRUNE, 784 S VILLIER CT., VIRGINIA BEACH, VA, 23452, US

(21) Appl. No.: 11/804,441

(22) Filed: May 18, 2007

(51) Int. Cl. A61B 18/20

(52) U.S. Cl. 606/9

(57) ABSTRACT

The method disclosed includes the combining of iontophoresis and ultrasound to deliver the appropriate chromophores to blonde, grey, white, vellus, and other non-pigmented or laser resistant hair follicles to facilitate photothermolysis hair removal using a laser, Intense Pulsed Light (IPL), a combination of laser and RF energy, or combination of IPL and RF.

This technique preferentially ultra-exposes the hair follicles to light energy which is absorbed preferentially by naturally and synthetic elements in and around the hair follicle. The result is focused photothermolysis damage resulting in death of the hair producing tissue.

The combined use of iontophoresis and ultrasound, the appropriate chromophore and laser light allows for immediate and permanent removal of unwanted hair in selected locations on the body of mammals. This combination is so effective that the patient can be treated with a single or at least minimal office visitations thereby eliminating the need to continuously prepare and re-prepare the skin with melanin or other chromophores prior to multiple—in office—laser or IPL treatments for completing desired hair removal results.

Additionally, this method discloses the use of iontophoresis together with High Intensity Focused Ultrasound (HIFU) and also Radio Frequency (RF) energy combined with High Intensity Focused Ultrasound (HIFU) to specifically target necrotizing energy doses into the regenerative zones of the hair and accomplish hair removal.

### FIELD OF INVENTION

The method disclosed includes the combining of iontophoresis and ultrasound to deliver the appropriate chromophores to blonde, grey, white, vellus, and other non-pigmented or laser resistant hair follicles to facilitate photothermolysis hair removal using a laser, Intense Pulsed Light (IPL), a combination of laser and Radio Frequency (RF) energy, or a combination of IPL and RF.

Additionally, this method discloses the use of iontophoresis together with High Intensity Focused Ultrasound (HIFU) and also Radio Frequency (RF) energy combined with High Intensity Focused Ultrasound (HIFU) to specifically target necrotizing energy doses into the regenerative zones of the hair to accomplish hair removal.

### BACKGROUND OF INVENTION

Historically, there have been a number of options for the permanent removal of hair. Electrolysis has been the most commonly selected approach, in which an operator, usually an electrologist, attaches an electrode to each individual hair shaft, with the patient typically holding a second electrode. An electrical current is then passed through the hair shaft and the hair follicle through the papilla. This precisely directed current can induce permanent injury in the follicle and papilla, stopping the future production of the hair shaft.

Problems exist with the electrolysis technique, however. The success with which hair is permanently removed varies greatly from patient to patient. Moreover, the process is slow since each hair follicle must be individually treated, and there is some discomfort associated with the electric current.

Other approaches have been proposed that rely on flashlamps, instead of lasers. This has the advantage of a less expensive, reasonably portable light source, but flashlamps create their own control problems. It is difficult to deliver light from the flashlamp to the skin, so the lamp must be placed in proximity to the skin. The reflectors that surround the flashlamp and collect the light and direct it to the skin must be precisely built and calibrated. Any error can cause hot spots in the spatial energy distribution. This can lead to under-treatment in some areas and burning in others. Moreover, the bandwidth of the light from these flashlamps is broad, usually including visible light and stretching into the longer infrared wavelengths. These longer wavelengths are well absorbed by water that dominates the skin make-up. Thus, the light from these sources tends to penetrate the skin very poorly, which leads to the use of higher fluence levels to sufficiently treat deeper-lying hair producing structures with the concomitant risk of burning or damaging the skin.

The removal of hair using lasers is another approach that has found success. Numerous techniques have been taught in the prior art. Each, however, suffers from drawbacks such as poor ultimate success in stopping hair growth even after multiple treatments, excessive injury to the tissue surrounding the hair follicles and papilla, and excessively large and expensive laser systems.

One approach relies on a pulsed laser source and the use of an exogenous absorber. A commercially available hair dye solution is first applied to the skin containing the unwanted hair and allowed to migrate along the hair shafts and into the follicles. The skin is then irradiated with a spot size of approximately 0.5 centimeters using a Q-switched YAG laser, or other short pulsed laser system. The pulse durations used by the lasers tend to be short, 15 nsec for the Q-switched laser. It appears that the sub-microsecond pulse durations shocks the hair follicle, which stops hair production, but only for a limited time. After months, the follicle again begins to produce hair, requiring further treatments or other techniques to yield any lasting success.

Still other approaches use laser light delivery systems that inject light into only a single hair follicle at a time. These have the advantage of a reduced concern for damaging tissue between hair follicles but have many of the same disadvantages associated with the electrolysis. That is, each individual hair and hair follicle must be separately treated.

Long pulse ruby lasers have recently been used in hair removal. The high energy ruby lasers, however, are generally large, inefficient types of laser light generators, where very long pulses are generated.

Photothermolysis as a method of hair removal can be divided into two general methods. The first being essentially the application of laser energy selected to be absorbed preferentially by elements residing naturally within the proximity of the hair follicle (see Zaias—U.S. Pat. No. 5,059,192). The second being differentiated from the first by introduction of a foreign element to provide a radiation target with an absorption frequency different from the naturally occurring elements (see Tankovich—U.S. Pat. Nos. 5,226,907 and 5,425,728). In both cases, death of the hair follicle is caused by localized thermolysis and subsequent destruction of the living tissue. One objective of any method of laser depilation is to minimize the radiation absorption of the surrounding skin and thereby reduce collateral damage.

A limitation of the second method, that of applying laser light by which natural skin and hair elements are not energized, is the obvious restriction from potential use of many available laser light sources. Because melanin naturally occurring in the skin is generally darker in color than the surrounding skin cells and tends to absorb, to some extent, a broad range of frequencies of incident light energy, depilation methods which attempt to avoid excitation of such natural elements are greatly limited.

In one study (Walther T., et. al., 1998) selective photothermolysis on dark pigmented hair follicles treated with a normal-mode ruby laser (694-nm wavelength, pulse duration 0.5 ms, fluence 20 J/cm², spot size 2 mm) was evaluated. For 15 volunteers, four test areas each were selected. After shaving, the first area was irradiated once, the second twice, the third three times and the fourth served as control area. A punch biopsy was taken from each volunteer immediately after the first laser treatment. Four weeks after the last irradiation, no effect was found in six cases and little effect in another six cases (50-90% regrowth). Hair regrowth of less than 30% was observed in only three cases. Eight weeks after the last session, no effect was found in 11 cases, little effect in 2 cases (10%) and less than 30% regrowth in only 2 cases. Twelve weeks after the last treatment, no difference could be detected between the areas untreated and those treated by laser. The laser parameters applied in this study did not result in effective epilation of body hair. In some cases, a delay in growth of several weeks was noticed.

### DEFINITIONS

Iontophoresis is a non-invasive method of propelling high concentrations of a charged substance, normally medication or bioactive-agents, transdermally by repulsive electromotive force using a small electrical charge applied to an iontophoretic chamber containing a similarly charged active agent and its vehicle. To clarify, one or two chambers are filled with a solution containing an active ingredient and its solvent, termed the vehicle. The positively charged chamber, termed the anode will repel a positively charged chemical, and the negatively charged chamber, termed the cathode, will repel a negatively charged chemical into the skin. Iontophoresis is well classified for use in transdermal drug delivery. Unlike transdermal patches, this method relies on active transportation within an electric field. In the presence of an electric field electromigration and electro osmosis are the dominant forces in mass transport.

There are a number of factors that influence iontophoretic transport including skin pH, drug concentration and characteristics, ionic competition, molecular size, current, voltage, time applied and skin resistance. Studies suggest that comparable iontophoretic doses delivered at low currents over longer periods are more effective than those delivered by high currents over a short periods.

The isoelectric point of the skin is ˜4; therefore, under physiological conditions, with the surface of the skin also buffered at or near 7.4, the membrane has a net negative charge and electro osmotic flow is from anode (−) to cathode (+). The phenomenon of electro osmosis has been used as a means to augment the anodic delivery of (in particular) large, positively charged drugs, the transport numbers of which are often extremely small (and whose iontophoretic enhancement therefore depends heavily upon electro osmosis) and to promote the transdermal migration of uncharged, yet polar, molecules, the passive permeation of which is typically very small.

The application of a charge to the skin alters the skin's permeability increasing migration of the active ingredient into the epidermis. There are a number of pathways that the ingredients could take, but research suggests that the majority of drugs permeate the skin via appendageal pores, including hair follicles and sweat glands, although some delivery is via the paracellular channels and minimal quantities are transcellular.

Transport of lipophilic drug molecules is believed to be facilitated by its dissolution into the lipid matrix of the stratum corneum however hydrophilic drugs which are thought to permeate through the open pores or cutaneous appendages (hair follicle and sebaceous glands) only accounts for 0.1% of the total skin surface area.

The various mentioned methods for hair removal require repeated treatments and have not been particularly successful in permanently preventing the growth of hair. Therefore it is desirable to have a method and system to treat blonde, grey, white, vellus, laser resistant and residual hair such that a minimal amount of treatments are performed and such that the treatment does not damage adjacent skin, and permanently inhibits hair growth.

### DESCRIPTION OF PRIOR ART

U.S. Pat. No. 6,936,044 to McDaniel, David H., assigned to Light BioScience, LLC., describes a method for stimulating hair growth comprising exposing a hair growth structure to a source of narrowband multichromatic electromagnetic radiation having a dominant emissive wavelength of from about 390 nm to about 1600 nm without having applied a drug, cosmeceutical, and/or chromophore to the hair growth structure. Then photostimulating the hair growth structure by maintaining the exposure of the hair growth structure to the source of narrowband multichromatic electromagnetic radiation for a clinically effective duration and at a clinically effective light intensity to stimulate hair growth without causing skin ablation.

U.S. Pat. No. 6,676,655 to McDaniel, David H., assigned to Light BioScience, LLC., describes a method for the manipulation of collagen, fibroblast, and fibroblast-derived cell levels in mammalian tissue comprising exposing tissue to a plurality of pulses from at least one source of narrowband, multichromatic electromagnetic radiation having a dominant emissive wavelength of from about 300 nm to about 1600 nm and filtering the source of narrowband, multichromatic electromagnetic radiation to regulate the transmission of infrared radiation to the tissue. The pulses have a duration of from about 0.1 femtoseconds to about 100 seconds, with the interpulse delay between the pulses from about 0.1 to about 1000 milliseconds and the energy fluence received by the tissue is less than about 10 joule per square centimeter. The source of narrowband, multichromatic electromagnetic radiation emits radiation in a bandwidth of about +/−100 nm around the dominant emissive wavelength.

U.S. Pat. No. 6,632,218 to Furumoto, et. al., assigned to Cynosure, Inc., describes a method of hair removal comprising performing multiple treatment sessions where each treatment session comprises the steps of: generating laser light having a wavelength of 710-810 nm and irradiating hair-bearing skin with the laser light with a series of discrete subpulses having a fractional duty cycle over an effective pulse duration of greater than 10 msec to heat a hair-producing structure in a stepwise manner and waiting at least several weeks between each treatment session. The method further comprises prior to irradiating the hair-bearing skin applying an index matching topical application to facilitate the coupling of the laser light deeply into the hair-bearing skin.

U.S. Pat. No. 6,398,753 to McDaniel, David H., unassigned, describes a method for at least one of stimulating cellular activity and inhibiting cellular activity comprising exposing at least one cell to light and ultrasound and applying an active agent to the at least one cell. The step of exposing at least one cell to light and ultrasound comprises exposing at least one cell to light and ultrasound to heat the cell to a specified temperature and applying an active agent to at least one cell.

U.S. Pat. No. 6,283,956 to McDaniel, David H., unassigned, describes a process for producing preferential damage to hair exiting mammalian skin through a hair duct the hair growing in a hair follicle and having a hair shaft and a hair bulb the process comprising selecting an agent having an average diameter for enabling the agent to penetrate the hair duct therein. The agent comprises a material for at least one of bonding to and becoming physically incorporated into at least one of the hair shaft, the hair follicle, the hair bulb and the hair duct, the agent having an electromagnetic radiation absorption characteristic for enabling the agent to absorb at least a first wavelength of electromagnetic radiation from a skin-penetrating electromagnetic radiation source. The agent is applied to the skin so that it penetrates the skin and at least one of bonds to and becomes physically incorporated into at least one of the hair shaft, the hair follicle, the hair bulb and the hair duct, and exposing the agent to at least a first wavelength of electromagnetic radiation, whereby the agent absorbs the first wavelength of electromagnetic radiation.

U.S. Pat. No. 5,871,479 to Furumoto, et. al., assigned to Cynosure, Inc., describes a hair removal laser system comprising a long pulse alexandrite laser which generates a laser light output pulse having a duration between 5 and 50 msec or longer, a wavelength between 710 and 800 nm, and a fluence between 10 and 50 J/cm². The alexandrite laser comprises an alexandrite gain medium with at least one flashlamp which is adapted to pump the alexandrite gain medium, a power supply which is connected to provide power to at least one flashlamp and an active pulse forming network which comprises at least two parallel-connected transistors that switch at least one flashlamp across the power supply and a light delivery system which transmits the laser light output pulse to a hair-bearing skin of a patient. The system further comprises an index matching topical application that is applied to the skin to facilitate the coupling of the laser light output pulse deeply into the skin.

U.S. Pat. No. 6,610,052 to Furumoto, et. al., assigned to Cynosure, Inc., describes a method for treating biologic tissue with laser light comprising generating a long effective laser light output pulse comprising a series of sub-pulses having a duty cycle that is less than 50% over a selected effective pulse duration and a periodicity that is less than the thermal relaxation time of a targeted structure and delivering the laser light to the tissue of a patient.

U.S. Pat. No. 6,080,127 to Li, et. al., assigned to AntiCancer, Inc, describes a method to improve delivery of an agent such that the agent is delivered to hair follicles. The method comprises the steps of administering the agent to skin containing hair follicles and applying vibration of from about 1 Hz to about 100 Hz with an amplitude of about 0.1 mm to about 10 mm in combination with the agent. The agent is applied with an apparatus that comprises a vibration means for applying vibration to the skin with a frequency of from about 1 Hz to about 100 Hz and an amplitude of about 0.1 mm to about 10 mm and a dispensing means for dispensing and applying the agent to the skin in combination with vibration.

U.S. Pat. No. 5,830,177 to Li, et. al., assigned to AntiCancer, Inc., describes a method to improve delivery of an agent to hair follicles comprising the steps of administering the agent to an area of skin containing hair follicles, and applying vibration of from about 1 Hz to about 100 Hz with an amplitude of about 0.1 mm to about 10 mm in combination with the agent. The agent is selected from the group consisting of minoxidil, substance-P, fenesteride, cyclosporin, melanin, tyrosinase, p21 protein, cell cycle inhibitors, cell proliferation inhibitors, anti-androgen agents and inhibitors of 5-α reductase.

U.S. Pat. No. 6,190,315 to Kost, et. al., assigned to Sontra Medical, Inc., describes a method for enhanced transport of a molecule into or through skin comprising the steps of permeabilizing the skin by applying low frequency ultrasound to the area of the skin where permeabilization is desired, wherein the frequency of the ultrasound is between about 20 kHz and 1 MHz and subsequently applying a driving force to extract analyte molecules through or to deliver drug molecules into the skin over a period of time following the permeabilization without repermeabilizing the skin.

U.S. Pat. No. 5,947,921 to Johnson, et. al., assigned to MIT, describes a method for enhancing transdermal transport of compounds comprising administering to the skin an effective amount of ultrasound in combination with linoleic acid in an ethanol solution.

U.S. Pat. No. 4,078,052 to Papahadjopoulos, P. Demetrios, assigned to US Dept of Health, Education & Welfare, describes a method for preparing large unilamellar phosphatidylserine vesicles with diameters of about 2,000-12,000 A which comprises subjecting a solution of phosphatidylserine in aqueous NaCl buffer to ultrasound treatment to give a sonicated preparation of the phosphatidylserine containing small spheroidal vesicals in the range of 200-500 A diameter, adding Ca2+ in the amount of 1-10 mM and incubating for about 30 minutes to 1 hour at room temperature above 10° C. to produce large multilamellar structures, a substantial portion of which are cochleate cylinders; and adding a chelating agent for Ca2+ at a pH of about 7.4 in an amount sufficient to chelate surplus calcium to produce the desired large closed spherical unilamellar phosphatidylserine vesicles.

U.S. Pat. No. 4,780,212 to Kost, et. al., assigned to MIT, describes a method comprising controlling transport of preselected species of molecules in a solution through a membrane by providing a membrane containing apparatus, contacting a solution containing molecules with the membrane, exposing the membrane containing apparatus to ultrasound, and adjusting the intensity, frequency and time of exposure of the ultrasound sufficient to selectively pass the preselected species of molecules through the membrane.

U.S. Pat. No. 6,398,753 to McDaniel, David H., unassigned, describes a method for at least one of stimulating cellular activity and inhibiting cellular activity comprising exposing at least one cell to light and ultrasound, and applying an active agent to the at least one cell.

U.S. Pat. No. 5,814,599 to Mitragotri, et. al., assigned to MIT, describes a method for enhancing delivery of a drug across the skin comprising applying the drug encapsulated in a liposome or polymeric microparticle to the skin in a pharmaceutically acceptable carrier having an absorbance coefficient equivalent to water and applying ultrasound at a frequency of between 20 kHz and less than 10 MHz at an intensity not causing any irreversible skin damage for a period of time effective to deliver the drug encapsulated in the liposome or microparticle across the skin in a desired drug dosage.

U.S. Pat. No. 6,808,720 to Unger, Evan C., assigned to ImaRx Therapeutics, Inc., describes a composition for use in the delivery of bioactive agents to a patient comprising cochleate vesicles under 2 μm in size. The cochleate vesicles comprise a charged lipid, a counter ion, a lipid covalently bonded to a polymer, and a bioactive agent, and wherein the composition further comprises a gas, a gaseous precursor, a gas and a gaseous precursor, a liquid perfluorocarbon compound or a liquid perfluoroether compound.

U.S. Pat. No. 6,030,374 to McDaniel, David H., unassigned, describes a method for enhancing the transport of an active agent through mammalian skin comprising exposing the skin to ultrasound, and applying an active agent to the skin, wherein the step of applying an active agent to the skin comprises injecting the active agent into the skin.

U.S. Pat. No. 6,629,971 to McDaniel, David H., unassigned, describes a process for stimulating growth of hair exiting mammalian skin through a hair duct, the process comprising selecting at least one of a photoactive agent and a photo sensitizing agent, the agent having an average diameter enabling the agent to penetrate the hair duct, the agent having an electromagnetic radiation absorption characteristic enabling the agent to absorb at least a first wavelength of electromagnetic radiation from a skin-penetrating electromagnetic radiation source applying the agent to the hair and skin, forming an agent-tissue complex by a process comprising reacting the agent with cells in the hair, the skin, or both, and exposing the agent-tissue complex to incoherent, multi-frequency electromagnetic radiation comprising at least the first wavelength of electromagnetic radiation, whereby the agent-tissue complex absorbs the first wavelength of electromagnetic radiation.

U.S. Pat. No. 6,586,000 to Luo, et. al., assigned to Dermatrends, Inc., describes a method for enhancing the flux of a drug through a body surface comprising administering the drug to a localized region of a human patient's body surface in combination with a hydroxide-releasing agent. The hydroxide-releasing agent is present in an amount effective to enhance the flux of the drug through the localized region of the body surface without causing damage thereto and effective to provide a pH in the range of approximately 8.5 to 13.0 at the localized region of the body surface during drug administration wherein the drug and hydroxide-releasing agent are present in a formulation and the amount of hydroxide-releasing agent in the formulation applied to the body surface is the total of the amount required to neutralize any acidic species in the formulation plus an amount equal to approximately 0.5 wt. % to 25.0 wt. % of the formulation.

U.S. Pat. No. 6,143,287 to Ben-Hur, et. al., assigned to New York Blood Center, describes a method for removing hair from a selected skin area comprising the steps of applying a liposome composition comprising a phthalocyanine to the selected skin area so that the composition is introduced into hair follicle ducts of the skin area, wherein the phthalocyanine is present in the composition in an amount effective to undergo a reaction and damage the hair follicles upon light activation of the phthalocyanine, removing from the skin area substantially all of the liposome composition which is not introduced into the hair follicles; and applying light to the skin area at the appropriate wavelength, energy, and duration to penetrate the skin and cause the phthalocyanine to undergo a reaction to damage the hair follicles; wherein the light is applied for about 5 minutes to about 6 hours, at a wavelength between about 500 and about 900 nm, and at an energy between about 20 and about 800 J/cm2 skin area.

U.S. Pat. No. 6,066,316 to Shiojima, et. al., assigned to Shiseido Co. Inc., describes a composition of wax comprising an amphoteric surfactant and optionally a semi-polar surfactant, a nonionic surfactant and a wax. The composition is in a transparent liquid phase which is characterized by having a weighted average HLB of 6 to 15 for the total nonionic surfactant, a weight ratio of amphoteric surfactant/(amphoteric surfactant+nonionic surfactant) of 0.03 to 0.5 and wherein the nonionic surfactant is a polyoxyethylene polyoxypropylene alkyl ether having a formula and optionally a different formula: wherein R is an alkyl group of an alkyl group having a carbon number from 12 to 24, m is in the range of 5<=m<=30, and n is in the range of 0<=n<=5.

U.S. Pat. No. 5,059,192 to Zaias, Nardo, unassigned, describes a method of hair depilation, comprising the steps of aligning a laser light applicator substantially vertically over a hair follicle opening, the applicator having an aperture of sufficient area to surround a hair follicle and overlie its papilla, applying through the aperture to the hair follicle a pulse of laser energy of a wavelength which is readily absorbed by the melanin of the papilla and having a radiant exposure dose of sufficient energy and duration to damage its papilla so that hair regrowth is prevented and scarring of the surrounding skin is avoided.

U.S. Pat. No. 5,226,907 to Tankovich, Nikolai I., unassigned, describes a process for the permanent destruction of plurality of hairs growing on a section of human skin comprising the steps of applying to the hairs and skin section a contaminant having a high absorption of at least one frequency band of light which penetrates human skin, cleaning the skin leaving at least a portion of the hairs under the skin contaminated with the contaminant, illuminating the skin section with at least one frequency band of light, a significant portion of which is absorbed in the contaminant so as to permanently destroy the plurality of hair.

U.S. Pat. No. 5,425,728 to Tankovich, Nikolai I., unassigned, describes a process for the permanent removal, from a section of human skin, of a plurality of hairs growing in hair ducts from follicles at the bottom of the ducts and being nourished by skin tissue immediately surrounding the follicles essentially without damage to skin tissue except to the skin tissue immediately surrounding the follicles, comprising the steps of selecting a contaminant capable of infiltrating the hair ducts and having a high optical absorption of at least one frequency band of light which will penetrate the section of skin, applying the contaminant to the surface of skin in such a manner as to cause a quantity of the contaminant to infiltrate the hair ducts, at least partly removing the contaminant from the section of skin but leaving at least a portion of the contaminant in the hair ducts and illuminating the section of skin with the at least one frequency band of light, a significant portion of which penetrates the section of skin and is absorbed in a quantity of contaminant in the hair ducts, the quantity of contaminant in the ducts and the energy absorbed in it being sufficient to cause a reaction which destroys the hairs by causing death of the follicles or of the skin tissue feeding the follicles.

U.S. Patent Publication No. 20060276741A1 to Henley, Julian L., unassigned, describes an ionosonic intradermal drug delivery device adapted to releasably attach to a person's body so that a skin-contacting surface of the device is adjacent to a targeted portion of the person's skin or nail. The device is operable for ionosonically driving a medicament across the skin-contacting surface of the device into the targeted portion of the person's skin or nail and the device comprises, in combination: a medicament carrying layer in fluid communication with the skin-contacting surface of the device comprising a porous sheet impregnated with a medicament containing fluid and an iontophoresis electrode in electrical communication with the medicament carrying layer providing means for iontophoretically driving the medicament into the targeted portion of the person's skin or nail(s) wherein the iontophoresis electrode comprises a plurality of electrode channels, each electrode channel of the plurality of electrode channels being electrically isolated from other electrode channels, the plurality of electrode channels having electrical connection means connected and thereto adapted for simultaneous electrical connection of selected electrode channels to the same or different current sources. Each electrode channel is in electrical communication with the medicament carrying layer and at least one piezoelectric element affixed to the iontophoresis electrode and overlying at least one of the iontophoresis electrode channels.

U.S. Patent Publication No. 20060167403A1 to Henley, Julian L., assigned to BioPhoretic Therapeutic Systems, LLC., describes a method of treatment by electrokinetic self-administration of a medicament into a treatment site for an individual comprising providing an applicator having a self-contained power supply, a first electrode on one side of the applicator and a ground electrode on one side of the applicator spaced from the first electrode. The first electrode, ground electrode and power supply being electrically coupled to one another. Applying the applicator to the treatment site with the first electrode overlying the treatment site and the ground electrode spaced from the treatment site in electrical contact with the individual's body and completing an electrical circuit through the individual's body, medicament or a carrier therefor interposed between the first electrode and the treatment site, whereby the first electrode and the ground electrode electrokinetically drives the medicament into the treatment site.

U.S. Patent Publication No. 20060265030A1 to McDaniel, David H., assigned to Light BioScience, LLC., describes a method for stimulating, inhibiting, or regulating gene expression, comprising exposing a living cell comprising at least one gene to a source of narrowband, multichromatic electromagnetic radiation.

U.S. Patent Publication No. 20060129209A1 to McDaniel, David H., assigned to Light BioScience, LLC., describes a method for stimulating hair growth comprising exposing a hair growth structure to a source of narrowband multichromatic electromagnetic radiation having a dominant emissive wavelength of from about 390 nm to about 1600 nm without having applied a drug, cosmeceutical, and/or chromophore to the hair growth structure. Then photostimulating the hair growth structure by maintaining the exposure of the hair growth structure to the source of narrowband multichromatic electromagnetic radiation for a clinically effective duration and at a clinically effective light intensity to stimulate hair growth without causing skin ablation.

U.S. Patent Publication No. 20030060811A1 to McDaniel, David H., assigned to Light BioScience, LLC., describes a process for producing preferential damage to hair exiting mammalian skin through a hair duct, the hair growing in a hair follicle and having a hair shaft and a hair bulb, the process comprising selecting an agent having an average diameter for enabling the agent to penetrate the hair duct. The agent comprises a material for at least one of attaching to and becoming physically incorporated into at least one of the hair shaft, the hair follicle, the hair bulb and the hair duct wherein the agent has an electromagnetic radiation absorption characteristic for enabling the agent to absorb at least a first wavelength of electromagnetic radiation from a skin-penetrating electromagnetic radiation source. The agent is applied to the skin so that the agent penetrates the skin and attaches to and becomes physically incorporated into at least one of the hair shaft, the hair follicle, the hair bulb and the hair duct and exposing the agent to at least a first wavelength of electromagnetic radiation whereby the agent absorbs the first wavelength of electromagnetic radiation.

U.S. Patent Publication No. 20040024390A1 to Furumoto, Horace W, assigned to Cynosure, Inc., describes a method for treating biologic tissue with pulse light comprising generating a long effective output light pulse comprising a series of sub-pulses having a fractional duty cycle over a selected effective pulse duration, a periodicity that is less than the thermal relaxation time of a targeted structure and an interpulse-delay between successive sub-pulses that is greater than the thermal relaxation time of non-targeted structures within the treatment area and delivering the output light to the tissue of a patient.

U.S. Patent Publication No. 20050191252A1 to Mitsui, Yukio, unassigned, describes a skin beautification cosmetic system comprising an ultrasonic vibrator using high frequency, an iontophoresis device, and a cosmetic additive.

U.S. Patent Publication No. 20020120225A1 to McDaniel, David H., unassigned, describes a method for enhancing the transport of an active agent through mammalian skin comprising exposing the skin to ultrasound, and applying an active agent to the skin. The step of applying an active agent to the skin comprises injecting the active agent into the skin, and wherein the active agent comprises at least one of Vitamin C, Vitamin E, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, an antioxidant, a phytoanthocyanin, a phytonutrient, a botanical product, a herbaceous product, a hormone, an enzyme, a mineral, a genetically engineered substance, a cofactor, a catalyst, an antiaging substance, insulin, trace elements, minerals, Rogaine, a hair growth stimulating substance, a hair growth inhibiting substance, a dye, a natural or synthetic melanin, a metalloproteinase inhibitor, proline, hydroxyproline, an anesthetic substance.

U.S. Patent Publication No. 20010041856A1 to McDaniel, David H., assigned to Alex Chartove, describes a method for at least one of stimulating cellular activity and inhibiting cellular activity comprising exposing at least one cell to at least one of light and ultrasound, and applying an active agent to the at least one cell.

U.S. Patent Publication No. 20030072724A1 to Maibach, et. al., unassigned, describes a method of treating an individual afflicted with skin hyperpigmentation comprising topically administering to a localized region affected by skin hyperpigmentation on the individual's body surface a formulation comprised of an active agent effective in treating skin hyperpigmentation, a pharmaceutically acceptable topical carrier, and a permeation-enhancing base, the base being present in a predetermined amount effective to enhance the flux of the active agent through the localized region of the body surface without causing damage thereto.

U.S. Patent Publication No. 20050129722A1 to Skold, Thomas, assigned to CollaGenex Pharmaceuticals, Inc., describes a water-based topical delivery system for an active substance, capable of enhancing skin barrier restoration in the stratum corneum comprising water; fatty acids; cholesterol; and a ceramide/phospholipid.

U.S. Patent Publication No. 20030077301A1 to Maibach, et. al., unassigned, describes a method of treating an individual afflicted with an inflammatory dermatosis comprising topically administering to a localized region affected by the inflammatory dermatosis on the individual's body surface a formulation comprised of an active agent effective in treating the inflammatory dermatosis, a pharmaceutically acceptable topical carrier, and a permeation-enhancing base, the base being present in a predetermined amount effective to enhance the flux of the active agent through the localized region of the body surface without causing damage thereto.

U.S. Patent Publication No. 20020034554A1 to Hsu, et. al., unassigned, describes a permeation enhancer composition comprised of a hydroxide-releasing agent and a lipophilic co-enhancer having a molecular weight in the range of about 150 to 1000 and an aqueous solubility of less than about 1 wt. %.

### SUMMARY OF THE INVENTION

It is an objective of the present disclosure to provide an improved method of permanent hair removal which employs iontophoresis and ultrasound or High Intensity Focused Ultrasound (HIFU) simultaneously or in conjunction with transdermal delivery of agents that may be delivered in pure form, in solution, as emulsions, within liposomes, within synthetic or natural microspheres, polymeric nanoparticles, microparticles, microcapsules, or other known micro encapsulation vehicles. Agents may include, without limitation, natural or synthetic melanin, aminolevulinic acid (ALA), hair dyes, vegetable dyes, food coloring, fabric dyes, tissue stains, shoe or leather dyes, and other plant products such as flavonols, cholorophyll, caroteniods, enzymes, etc.

Agents may be delivered in pure form, in solution, in emulsions, in liposomes, in synthetic or natural microspheres, polymeric nanoparticles, microparticles, microcapsules, or other known micro encapsulation and include a topical gel, lotion, liquid, cream or ointment composition that may also include melanin encapsulated liposomes. The agent is designed to attach to, or become physically incorporated into, the hair shaft, the hair follicle, the hair bulb, the hair duct and or papilla.

The ultrasound energy causes break up of the top most surface of the skin and allows the melanin encapsulated liposome composition to migrate into the hair follicle. This technique preferentially ultra-exposes the hair follicles to light energy which is absorbed preferentially by naturally occurring and synthetic elements in and surrounding the hair follicle. The result focuses photothermolysis damage resulting in death of the hair producing tissue. Melanin is one of the most common chromophores found in the hair follicle used for the selective photothermolysis of unwanted hair, and other chromophores such as ALA can be used also. Laser or other intense pulsed light between 100 and 1600 nm is appropriate for targeting melanin and ALA in the hair shaft.

Normal human hairs can be classified according to the 3 phases of their growth cycle: anagen, catagen, and telogen. Anagen hairs are in a growing phase, during which the matrix cells of the hair follicle undergo vigorous mitotic activity.

Toward the end of the anagen phase, the amount of pigment decreases at the base of the follicle, which expands to form a keratinized club. Then, the hair enters the catagen phase, a transitional phase in which mitotic activity decreases. The follicle separates from the dermal papilla and the capillary plexus and moves upward within its connective tissue sheath toward the epidermis. The resulting telogen hairs, or club hairs, are in a resting phase. These hairs have short, club-shaped roots that anchor them in the follicle. They lack root sheaths and show depigmentation of the proximal part of the shaft.

The hairs continue in this resting state until the follicle spontaneously reenters the anagen phase. At this point, the club hairs are forced out by growing hairs underneath them, and the cycle begins anew. The cycle is not synchronous throughout the scalp; patches of hair may be synchronized. The length of each phase of the cycle, as well as the length of the entire cycle, varies with the site and the age of the patient. In the scalp, for example, the average length of the anagen phase is 1000 days; that of the catagen phase, only a few days; and that of the telogen phase, 100 days. Of the 100,000 hairs on the average scalp, 10-15% are in the catagen or telogen phase at any time. Most hair follicles are in the anagen stage at any given point in time.

The combined use of melanin encapsulated liposomes or other chromophores in conjunction with iontophoresis and ultrasound or iontophoresis and HIFU followed by a treatment using a laser, IPL, a combination of laser and RF energy, or a combination of IPL and RF allows for immediate and permanent removal of unwanted hair in selected locations on the body of mammals. This combination is so effective that the patient can be treated with a single or at least minimal office visitation thereby eliminating the need to continuously prepare and re-prepare the skin with melanin or other chromophores prior to multiple—in office—laser treatments for completing desired hair removal results primarily in removing hairs that are primarily in the anagen phase.

It is another object of the disclosure to provide a composition where the liposomes have a size range of 1 nanometer (nm) to 100 microns.

It is another object of the disclosure to provide a method of hair removal which increases the quantity of melanin or other chromophores surrounding the hair follicle to create an enhanced target for incident light energy and improve the preferential absorption in the proximity of the hair follicle.

It is another object of the disclosure to provide an improved method of laser hair removal which uses laser, IPL, a combination of laser and RF energy, a combination of IPL and RF or other light energy of a wavelengths or multiple wavelengths which are absorbed by naturally occurring or synthetic elements such as melanin in the skin or hair.

It is also an object of the disclosure to provide a method of destroying skin cancer cells by photothermolysis by selective delivery of melanin using liposomes to create an enhanced target for subsequent application of light energy, which

minimizes required irradiation and thereby minimizes the damage to the surrounding skin.

It is a further object of the disclosure that a variety light energy sources, both coherent and incoherent, including such lasers as ruby red and Nd-YAG, alexandrite, and diode lasers, or any other light source providing an effective frequency and intensity.

It is also another object of the disclosure that the light energy should be in the wavelength range of 100 to 1600 nm.

For the ultrasound application, High Intensity Focused Ultrasound (HIFU) can be provided in an innovative and cost-effective hair-removal appliance for cosmetic use.

The ultrasonic energy integrates low-cost piezoelectric crystals and a pioneering method for the production of HIFU.

This proprietary technology (patent pending) is utilized to specifically target necrotizing energy doses into the regenerative zones of the hair. Applisonix's ultrasonic design is expected to make its use familiar, friendly and safe for both professionals and common domestic users.

The technology is expected to be significantly safer than laser/IPL, because the energy is applied directly into the hair shaft and not irradiated over large skin surfaces (such as in laser and IPL treatments). Likewise, counter to the technological limitations of laser/IPL, this ultrasound technology is effective regardless of hair color or skin tone.

It is also an object of the disclosure to provide a more effective method of destroying regenerative zones of the hair by combining RF energy with HIFU to provide a more effective treatment.

It is also an object of the disclosure to provide a more effective method of destroying regenerative zones of the hair by combining Iontophoresis with HIFU to provide a more effective treatment that either combination alone.

The combined use of iontophoresis and ultrasound, the appropriate chromophore and laser light allows for immediate and permanent removal of unwanted hair in selected locations on the body of mammals. This combination is so effective that the patient can be treated with a single or at least minimal office visitations thereby eliminating the need to continuously prepare and re-prepare the skin with melanin or other chromophores prior to multiple—in office—laser or IPL treatments for completing desired hair removal results.

Additionally, this method discloses the use of iontophoresis together with High Intensity Focused Ultrasound (HIFU) and also Radio Frequency (RF) energy combined with High Intensity Focused Ultrasound (HIFU) to specifically target necrotizing energy doses into the regenerative zones of the hair and accomplish hair removal.

### BRIEF DESCRIPTION OF THE DRAWINGS

FIG. 1 is an illustration that indicates how the method of combining iontophoresis and ultrasound is effective in driving particles into and around the hair shaft, follicle and papilla to provide a target sufficient for effective laser hair removal.

### DETAILED DESCRIPTION OF THE DRAWINGS

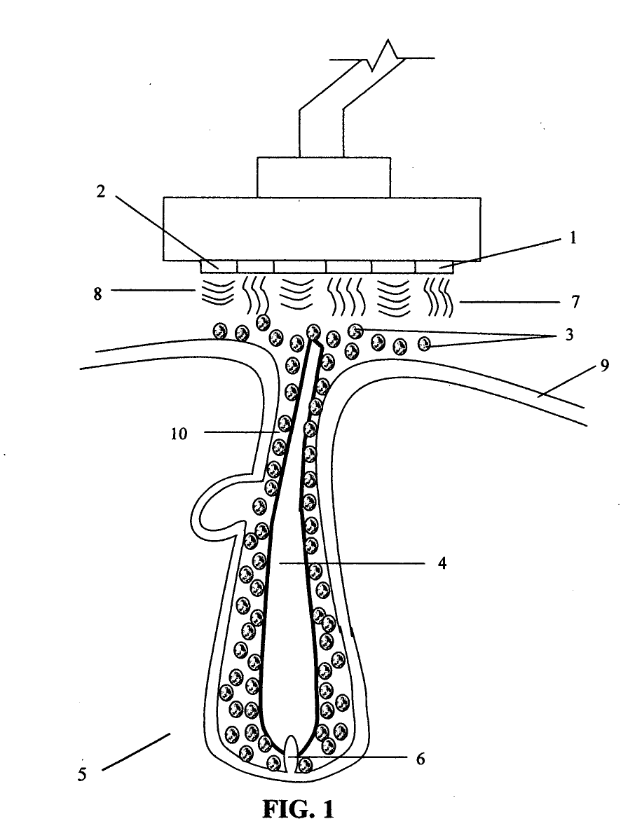

FIG. 1 is an illustration how the method of combining iontophoresis [1] and an ultrasound or HIFU generator [2] is effective in driving target agents [3] into and around the hair shaft [4], hair follicle [5] and papilla [6] to provide a sufficient amount of target agents [3] for effective laser hair removal (not shown). The target agents [3] are topically applied to the epidermis [9] near the hair shaft [4] to be removed.

An electrical current [7] produced by iontophoresis [1] as well as ultrasound waves [8] produced by an ultrasound generator [2] are generated simultaneously working in conjunction to assist with carrying the target agents [3] from the epidermis [9] into the hair duct [10] and hair follicle [5] surrounding the hair shaft [4]. The target agents [3] may be any of or a combination of natural or synthetically derived melanin, ALA, hair dyes, vegetable dyes, food coloring, fabric dyes, tissue stains, shoe leather dyes, and other plant derived products including flavanols, chlorophyll, carotenoids, and enzymes. Additionally the target agents [3] may be a topical gel, lotion, liquid, cream or ointment formulation and also comprising melanin encapsulated liposomes. Movement of the target agents [3] into the hair follicle [5] causes the hair shaft [4] to become physically incorporated with the target agents [3].

Selection of the target agents [3] to be used is dependent on the type and laser frequency, or photothermolysis, being used for hair removal. In a preferred mode the laser frequency matches the absorptive frequency of the target agent [3]. As the hair follicle [5] with the target agent [3] is exposed to the laser frequency, the hair shaft [4] and target agents [3] molecules begin to vibrate at their natural frequencies, generating heat and destroying the hair shaft [4], papilla [6], other structures in the follicle, and target agents [3].

1. A method of providing permanent hair removal employing a combination of; iontophoresis and ultrasound, IPL and laser or equivalent light energy, wherein said combination includes using iontophoresis and ultrasound or HIFU simultaneously for transdermally delivering natural or synthetically derived melanin, ALA, hair dyes, vegetable dyes, food coloring, fabric dyes, tissue stains, shoe leather dyes, and other plant derived products optionally including; flavanols, chlorophyll, carotenoids, and enzymes, by using a topical gel, lotion, liquid, cream or ointment formulation, said method also comprising melanin encapsulated liposomes using ultrasound for causing break up of the top most surface of the skin allowing said melanin encapsulated liposomes composition or other chromophores to migrate into said skin directed toward hair follicles such that said liposome composition or other chromophores may reside in or become physically incorporated into, the hair shaft, the hair follicles, the hair bulb, or the hair duct so that using IPL, laser, a combination of laser and RF, a combination of IPL and RF or equivalent light energy results in preferential ultra-exposure of said hair follicles providing focus for photothermolysis to facilitate destroying hair producing tissue.

2. The method of claim 1, wherein said permanent hair removal can be provided in one or more patient treatments.

3. The method of claim 1, wherein said permanent hair removal is provided in a single treatment.

4. The method of claim 1, wherein melanin in the hair follicle is the chromophore for the selective photothermolysis of unwanted hair.

5. The method of claim 1, wherein a laser or other intense pulsed light between 300 to 1600 nm is appropriate for targeting melanin in a hair follicle shaft.

6. The method of claim 1, wherein removal employing a combination of;
iontophoresis, ultrasound or HIFU, and laser or equivalent light energy eliminates the need to continuously prepare and re-prepare the skin with melanin or other chromophores prior to multiple in office laser treatments for completing desired permanent hair removal.

7. The method of claim 1, comprising the step of exposing the skin to at least one enzyme prior to exposure to ultrasound and iontophoresis.

8. The method of claim 1, wherein removal of hair is primarily blond, white, grey, vellus, laser resistant, or residual hair.

9. The method of claim 1, wherein said pulse length for laser light is from about 1 nanosecond to about 1 second.

10. The method of claim 9, wherein said pulse length is from about 1 nanoseconds to about 100 milliseconds.

11. The method according to claim 1, wherein the laser or equivalent light energy fluence received at said hair producing tissue is 300 J/cm² or less.

12. The method according to claim 9, wherein the energy fluence received at said hair producing tissue is greater than 1 J/cm² and further comprises cooling said tissue.

13. The method according to claim 12, wherein said energy fluence received at said tissue is from about from about 1×10⁻⁶ J/cm² to 1 J/cm².

14. The method of claim 13, wherein said energy fluence received at said tissue is from about from about 1×10⁻³ J/cm² to about 0.1 J/cm².

15. The method of claim 14, further comprising cooling said tissue to maintain a temperature of said tissue below a threshold for thermal injury.

16. The method of claim 15, further comprising the step of maintaining the temperature of said tissue at or below 38 degrees Celsius.

17. A set of devices for providing permanent hair removal employing a combination of; iontophoresis and ultrasound, IPL and laser or equivalent light energy, wherein said combination includes using iontophoresis and ultrasound or HIFU simultaneously for transdermally delivering natural or synthetically derived melanin, ALA, hair dyes, vegetable dyes, food coloring, fabric dyes, tissue stains, shoe leather dyes, and other plant derived products optionally including; flavanols, chlorophyll, carotenoids, and enzymes, by using a topical gel, lotion, liquid, cream or ointment formulation, said method also comprising melanin encapsulated liposomes using ultrasound for causing break up of the top most surface of the skin allowing said melanin encapsulated liposomes composition or other chromophores to migrate into said skin directed toward hair follicles such that said liposome composition or other chromophores may reside in or become physically incorporated into, the hair shaft, the hair follicles, the hair bulb, or the hair duct so that using IPL, laser, a combination of laser and RF, a combination of IPL and RF or equivalent light energy devices results in preferential ultra-exposure of said hair follicles providing focus for photothermolysis to facilitate destroying hair producing tissue.

18. The devices of claim 17, wherein said permanent hair removal can be provided in one or more patient treatments.

19. The devices of claim 17, wherein said permanent hair removal is provided in a single treatment.

20. The devices of claim 17, wherein melanin in the hair follicle is the chromophore for the selective photothermolysis of unwanted hair.

21. The devices of claim 17, wherein said laser device or other intense pulsed light between 100 to 1600 nm is appropriate for targeting melanin in a hair follicle shaft.

22. The devices of claim 17, wherein removal employing a combination of; iontophoresis, ultrasound or HIFU, and laser or equivalent light energy eliminates the need to continuously prepare and re-prepare the skin with melanin or other chromophores prior to multiple in office laser treatments for completing desired permanent hair removal.

23. The devices of claim 17, wherein removal of hair is primarily blond, white, grey, vellus, laser resistant, or residual hair.

24. The devices of claim 17, wherein said pulse length for laser light from said laser device is from about 1 nanosecond to about 1 second.

25. The devices of claim 23, wherein said pulse length is from about 1 nanoseconds to about 100 milliseconds.

26. The devices according to claim 16, wherein the laser or equivalent light energy fluence received at said hair producing tissue is 300 J/cm² or less.

27. The devices according to claim 26, wherein the energy fluence received at said hair producing tissue is greater than 1 J/cm² and further comprises cooling said tissue.

28. The devices according to claim 27, wherein said energy fluence received at said tissue is from about from about 1×10⁻⁶ J/cm² to 1 J/cm².

29. The devices of claim 28, wherein said energy fluence received at said tissue is from about from about 1×10⁻³ J/cm² to about 0.1 J/cm².

30. The devices of claim 29, further comprising cooling said tissue to maintain a temperature of said tissue below a threshold for thermal injury.

31. The devices of claim 29, further comprising the step of maintaining the temperature of said tissue at or below 38 degrees Celsius.
